(19) United States
(12) Patent Application Publication
(10) Pub. No.: US 2004/0132110 A1
(43) Pub. Date: Jul. 8, 2004

(54) Method for diagnosing chronic intestinal inflammatory diseases

(76) Inventors: Pierre Desreumaux, Lille (FR); Jean Frederic Colombel, La Madeleine (FR); Laurent Dubuquoy, Eparcy (FR)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/473,480

(22) PCT Filed: Mar. 27, 2002
(86) PCT No.: PCT/FR02/01076

(30) Foreign Application Priority Data
Mar. 27, 2001 (FR) 01/04128

(51) Int. Cl. C12Q 1/68; G01N 33/53; G01N 33/567

(52) U.S. Cl. 435/7.2; 435/6

(57) ABSTRACT

The invention relates to a method for the in vitro diagnosis of chronic intestinal inflammatory diseases, such as Crohn's disease or haemorrhagic rectal colitis. The invention also relates to the use of compounds, such as an antibody, ligand or nucleic probe capable of specifically recognising PPARγ gene expression products, for the preparation of the composition or kit for diagnosing said diseases.

The present invention relates to a method for the in vitro diagnosis of chronic inflammatory bowel diseases, such as Crohn's disease or ulcerative colitis, and also to the use of compounds, such as an antibody, ligand or nucleic acid probe capable of specifically recognizing PPARγ gene expression products, for preparing a composition or a kit for diagnosing these diseases.

Crohn's disease (CD) and ulcerative colitis (UC) are chronic inflammatory bowel diseases (IBDs) which strike with predilection young adults, and which progress via attacks interspersed with periods of remission. They constitute one of the major problems of hepatogastroenterology.

In fact, these diseases strike young individuals and progress in a chronic or prolonged manner, potentially throughout life. These diseases can affect the entire digestive tube, from the mouth to the anus. These diseases also frequently have an effect on the personal and professional life, by virtue of the frequency of the attacks, the complications and sometimes the need for surgery. Since their cause is not known, there is no curative treatment for these diseases. Even though it is effective in the immediate, medical treatment only has the effect of interrupting the disease. In addition, the cost of such an interrupting medical treatment, which is already high, will increase with new medicinal products.

The prevalence of IBDs is increasing due to an incidence which was increasing up until the 1970s and has been stable since. On the basis of the incidence figures for 1991, and on the estimated median for survival, it has thus been estimated that, in 2005, among 232 million white Americans, 580 000 will be suffering from CD and 742 000 will be suffering from UC.

In France, the data from the EPIMAD register [register of chronic inflammatory diseases of the digestive tube] for the north-west of the country show a stable incidence since 1988 for IBDs: 5.6 for CD and 3.5 for UC. Using the same rules of calculation as in the United States, this gives an expected figure in France of 120 000 CDs and 80 000 UCs in 2005.

The diagnosis for IBDs is based on a set of clinical, morphological and histological criteria. The lesions during CD can reach all the segments of the digestive tube, whereas UC affects only the colon. The existence of isolated colonic lesions therefore represents a tricky situation for making the diagnosis of CD or UC or for differentiating an IBD from other pathologies associated with a bowel inflammation, such as, for example, bacterial or viral infections or infections with parasites, alternatively vascular pathologies, pathologies which are side effects of taking medicinal products, diverticulitis, etc.

Faced with the difficulty of establishing the diagnosis of CD or UC, the development of new diagnostic markers is of fundamental value and will make it possible to improve patient treatment.

Thus, there exists today a great need to have available an effective and reliable marker for chronic inflammatory bowel diseases, such as Crohn's disease or ulcerative colitis, such a marker having to be preferably easy to use and relatively inexpensive.

This is precisely the subject of the present invention.

Using Western blotting and immunohistochemistry techniques, the inventors have demonstrated, surprisingly, a deficiency in expression of the peroxysome proliferator-activated receptor PPARγ in the colonic mucosa of patients suffering from UC.

The inventors have thus been able to demonstrate, surprisingly, that the level of expression of the gene encoding the PPARγ receptor (PPARγ for Peroxysome Proliferator-Activated Receptor gamma) at the cell surface of cells of the epithelium and/or of cells of the lamina propria of the bowel tissue is correlated with the diagnosis of, or with the prognosis for, chronic inflammatory bowel diseases.

The inventors have also, and just as surprisingly, demonstrated that this level of expression of PPARγ, added to its distribution according to cell type, cells of the epithelium and/or cells of the lamina propria, makes it possible not only to refine this diagnosis or prognosis, but also to diagnose with precision, or to give a precise prognosis for, the type of chronic inflammatory bowel diseases of the patient tested.

The peroxysome proliferator-activated receptor PPARγ is a receptor which controls the expression of a large number of genes involved in metabolism (Schoonjans, et al., Curr. Opin. Lipidol., 8:159-166, 1997). In vitro studies have already shown that PPARγ activators limit the production of inflammatory mediators such as the inflammatory cytokines produced by human macrophagemonocytes (Ricote, et al., Nature, 391:79-82, 1998; Jiang, et al., Nature, 391:82-86, 1998) and by intestinal epithelial cells (Lefebvre, et al., Endocrinology, 162:331-340, 1999). The use, in vivo, of PPARγ agonists has also been described for the treatment of colitis induced in mice by administering sodium dextran sulfate (Su, et al., J. Clin. Invest., 104:383-9, 1999), or 2,4,6-trinitrobenzenesulfonic acid (Dubuquoy et al., Gastroenterol. Clin. Biol. 24:719-24, 2000).

Mention may also be made of:

the U.S. Pat. No. 5,861,274 published on Jan. 19, 1991, which describes the nucleic acid and peptide sequence of the human PPARγ receptor;

the publication by Desreumaux et al., 1999 (Gastoenterology, 117, 1, 73-81, 1999) which describes an aberrant expression of PPARγ in the mesenteric adipose tissue of patients suffering from CD and a deficiency in regulation of PPARγ by TNFα in the adipocyte;

the publication by Lefebvre et al., 1999 (J. of Endocrinology, 162, 3, 331-340, 1999), which describes the expression of PPARγ in the digestive tube in mice and humans;

the publication by Su Chinu et al., 1999 (J. of Clinical Investigation, 104, 4, 383-389, 1999) which describes the anti-inflammatory role of PPARγ agonists in vitro and in vivo in an experimental model of colitis in mice induced by sodium dextran sulfate;

the U.S. Pat. No. 6,166,049 published on Dec. 26, 2000, which describes the use PPARγ agonists in the treatment or prevention of syndrome X; and, finally

the two documents Hafraoui et al., 1999 (Gastroenterology, 116, 4, part 2, page A689, 1999 and Immunology Letters, 69, 1, 161-162, 1999), which describe a difference in expression of PPARγ mRNA in the course of UC vs CD and in control patients, quantitative PCR.

A subject of the present invention is therefore an in vitro method for diagnosing or for giving a prognosis for an inflammatory bowel disease in a patient, characterized in that it uses a step for comparing the amount of product from expression of the gene encoding PPARγ in a bowel tissue sample, said sample comprising at least cells of the intestinal epithelium and/or of the intestinal lamina propria, with respect to a control sample.

The present invention also relates to a method according to the invention, characterized in that said sample of bowel tissue comprises at least cells of the intestinal epithelium and of the intestinal lamina propria, and in that it also uses a step for comparing the distribution of the product from expression of said PPARγ gene between said cells of the intestinal epithelium and of the intestinal lamina propria on the patient's sample.

The above diagnostic methods according to the invention are preferably characterized in that they comprise the steps of the methods defined below.

The present invention thus relates to an in vitro method for determining the amount of product from expression of the gene encoding PPARγ (PPARγ gene) on a sample of bowel tissue, said sample comprising at least cells of the intestinal epithelium and/or of the intestinal lamina propria, characterized in that it comprises the following steps:

a) taking a sample of bowel tissue from a patient who may be suffering from or who may progress toward an inflammatory bowel disease, said sample comprising at least cells of the intestinal epithelium and/or of the intestinal lamina propria;

b) bringing the PPARγ gene expression product into contact with a compound capable of binding specifically to said PPARγ gene expression product, under conditions which allow the formation of a complex between said compound and said expression product, said compound being labeled or being capable of being labeled in order to obtain a signal representative of the amount of said expression product present in the sample and, where appropriate, making it possible to localize said expression product; and

c) quantifying or visualizing the signal obtained in step b).

The present invention also comprises a method according to the invention, characterized in that said sample of bowel tissue comprises at least cells of the intestinal epithelium and of the intestinal lamina propria, and in that it also uses a step for comparing the distribution of the PPARγ gene expression product between said cells of the intestinal epithelium and of the intestinal lamina propria on the patient's sample.

Preferably, said sample is a biopsy of bowel tissue taken from the patient's colon or small intestine, in particular of bowel tissue taken from the patient's colon.

In the methods according to the present invention, any conventional procedure or assay can be used to obtain, in step b) of said methods, a detectable and/or quantifiable signal representative of the amount of said expression product present in the sample, depending on the expression products sought, preferably the polypeptide corresponding to the product of translation of said PPARγ gene, in particular using the “Western blotting” method or using immunohistochemistry well known to those skilled in the art, or to its transcription product (mRNA) encoding said PPARγ receptor, in particular using the “in situ hybridization” method also well known to those skilled in the art, methods which will not be developed in the present description. Reference may, however be made to the examples below for the “Western blotting” method or the method by immunohistochemistry.

In a particularly preferred embodiment, the method according to the invention is characterized in that said product from expression of said PPARγ gene is the product of translation of said PPARγ gene, and in that the compound capable of binding specifically to said PPARγ gene expression product in step b) is chosen from the following compounds:

a monoclonal or polyclonal antibody, where appropriate labeled, directed against the PPARγ receptor, or one of its fragments; and

a natural or synthetic, where appropriate labeled, ligand which is an agonist or antagonist for the PPARγ receptor.

The expression “product of translation of said PPARγ gene” is intended to denote in particular the polypeptide corresponding to the PPARγ receptor expressed by the intestinal mucosa, in particular at the surface of cells of the intestinal epithelium and of the intestinal lamina propria.

The amino acid sequence of the human PPARγ receptor is in particular described in U.S. Pat. No. 5,861,274 (SEQ ID No. 2).

In general, for the preparation of monoclonal antibodies or their fragments directed against the PPARγ receptor, reference may be made to the techniques which are in particular described in the manual “Antibodies” (Harlow et al., Antibodies: A Laboratory Manual, Cold Spring Harbor Publications p. 726, 1988) or to the techniques for preparation from hybridomas described by Kohler and Milstein, Nature, 256:495-497, 1975.

The monoclonal or polyclonal antibodies which can be used in the methods according to the invention can be obtained, for example, using a cell of an animal immunized against the PPARγ protein, or one of its fragments, comprising the specific epitope (determinant of the protein responsible for the specific interaction with the antibody).

Said PPARγ receptor protein, or one of its fragments, may in particular be produced, according to usual procedures, by genetic recombination from a nucleic acid sequence contained in the sequence of the cDNA encoding the PPARγ receptor protein, or by peptide synthesis from an amino acid sequence included in the peptide sequence of the OZF protein.

The monoclonal or polyclonal antibody fragments according to the invention comprise any fragment of said monoclonal antibody capable of binding to the epitope of the PPARγ protein to which the monoclonal or polyclonal antibody from said fragment is derived binds. Examples of such fragments include in particular single-chain monoclonal or polyclonal antibodies or monovalent Fab or Fab′ fragments and divalent fragments such as F(ab′)₂, which have the same binding specificity as the monoclonal antibody from which they are derived. A fragment according to the invention may also be a single-chain Fv fragment produced by methods known to those skilled in the art and as described, for example, by Skerra, et al., Science, 240:1038-1041, 1988 and King et al., Biochemical J., 290:723-729, 1991.

The monoclonal or polyclonal antibody fragments of the invention can be obtained from the monoclonal or polyclonal antibodies as described above by methods such as digestion with enzymes, for instance pepsin or papain, and/or by cleavage of disulfide bridges by chemical reduction. In another way, the monoclonal or polyclonal antibody fragments can be synthesized by automatic peptide synthesizers such as those provided by the company Applied Biosystems, etc., or can be prepared manually using techniques known to those skilled in the art and as described, for example, by Geysen, et al., J. Immunol. Methods, 102:259-274, 1978.

The monoclonal or polyclonal antibodies, or their fragments, and also the specific ligands capable of binding specifically to said translation product can also, according to the invention, be in labeled form in order to obtain a detectable and/or quantifiable signal.

The antibodies, or their fragments, or said ligands which are labeled according to the invention include, for example, antibodies or ligands, termed immunoconjugates, which can be conjugated, for example, with enzymes such as peroxidase, alkaline phosphatase, P-D-galactosidase, glucose oxidase, glucose amylase, carbonic anhydrase, acetylcholinesterase, lysozyme, malate dehydrogenase or glucose-6-phosphate dehydrogenase, or with a molecule such as biotin, digoxigenin or 5-bromodeoxyuridine. Fluorescent labels can also be conjugated to the antibodies, or their fragments, or to said ligands, and include in particular fluorescein and its derivatives, fluorochrome, rhodamine and its derivatives, GFP (for Green Fluorescent Protein), dansyl, umbelliferone, etc. In such conjugates, the antibodies, or their fragments, or said ligands can be prepared by methods known to those skilled in the art. They can be coupled to the enzymes or to the fluorescent labels directly or via a spacer group or a binding group such as a polyaldehyde, for instance glutaraldehyde, ethylenediaminetetraacetic acid (EDTA) or diethylenetriaminepentaacetic acid (DPTA), or in the presence of coupling agents such as periodate, etc. The conjugates comprising labels of the fluorescein type can be prepared by reaction with an isothiocyanate.

Other conjugates may also include chemiluminescent labels, such as luminol and dioxetanes, or bioluminescent labels, such as luciferase and luciferin.

Among the labels which can be attached to the monoclonal or polyclonal antibody, or one of their fragments, or to said ligands, mention may also be made of radioactive labels which can be detected by known means, such as a gamma counter or a scintillation counter, by autoradiography, etc.

In the methods according to the present invention, any conventional procedure or assay can be used to obtain, in step b) of said methods, a detectable and/or quantifiable signal representative of the amount of said expression product present in the sample, in particular using Western blotting or using immunohistochemistry.

In these “Western blotting” or “immunohistochemistry” techniques, the specific complex formed between the PPARγ gene translation product and a compound capable of binding specifically to said translation product may be the result of bringing an antibody, or one of its fragments, or a ligand specific for the PPARγ receptor, into contact with said translation product.

In such a detection and/or quantification, said assay can be a competition or sandwich assay, or any assay known to those skilled in the art which depends on the formation of an antibody-antigen or ligand-receptor immunocomplex.

The antibody, or one of its fragments, or the ligand can also be immobilized. This immobilization may be carried out on many supports known to those skilled in the art. These supports may in particular include glass, polystyrene, polypropylene, polyethylene, dextran, nylon, or natural or modified celluloses. These supports may be either soluble or insoluble. Mention may in particular be made of solid supports such as polystyrene beads or microtitration plates, which are well known in the immunoassay field and which will not be developed in the present description.

By way of example, a preferred method uses immunoenzyme processes according to the ELISA technique, by immunofluorescence or immunoluminescence or radioimmunological technique (RIA), gold labeling or equivalent.

The techniques and the specific reagents making it possible to demonstrate, identify, localize and/or assay the antigen-antibody or ligand-receptor complexes, which may be used in the methods of the invention, may of course be combined, when the antibodies or ligands participating in the complex are not already immunoconjugated or labeled, with the appropriate reagents. These appropriate reagents will, for example, be immunoconjugated or labeled antibodies, as described above, capable of specifically recognizing the antibodies or ligands participating in said complex. The chromogenic substrates specific for the conjugated enzymes and the control reagents for positive, negative and quantitative control will also be combined with these techniques or reagents.

The expression “natural or synthetic ligand which is an agonist or antagonist” of the PPARγ receptor is here intended to denote any compound, other than an anti-PPARγ antibody, capable of binding specifically to the PPARγ receptor.

Among these ligands which are agonists or antagonists, preference is given to those chosen from natural agonists such as prostaglandin J2 or polyunsaturated fatty acids, and synthetic agonists such as thiazolinedione.

In a preferred embodiment, the method according to the invention is of the Western blotting type and is characterized in that, prior to step b):

the total proteins are extracted from the sample taken; and then

separated and transferred onto membrane (Western blotting); and

in that, in step c), the signal obtained in step b) is quantified.

The separation will in particular be carried out by acrylamide gel electrophoresis (PAGE), the transfer being carried out onto membranes well known to those skilled in the art, such as in particular nitrocellulose or PVDF membranes.

In a particularly preferred embodiment, the method according to the invention is of the “immunohistochemistry” type and is characterized in that, prior to step b):

a histological section of the sample taken is prepared, said sample taken having been, where appropriate, fixed in a solution of paraformaldehyde or formaldehyde (at approximately 4%) and embedded in paraffin; and

in that, in step c), the signal obtained in step b) is visualized.

The samples taken which, beforehand, have been fixed in a solution of paraformaldehyde or formaldehyde and embedded in paraffin may have been stored for several days or less at 4° C. or in frozen form.

In another aspect, the method according to the invention is characterized in that said product from expression of said PPARγ gene is the mRNA product of transcription of said PPARγ gene, and in that the compound capable of binding specifically to said PPARγ gene expression product in step b) is chosen from the nucleic acid sequences, where appropriate labeled, capable of hybridizing specifically (under high stringency conditions) with a fragment of said mRNA.

Among the methods for detecting (or localizing) and/or quantifying the mRNA of the PPARγ gene, the “in situ hybridization” method well known to those skilled in the art is preferred.

The nucleic acid sequences, where appropriate labeled, capable of hybridizing specifically (under high stringency conditions) with a fragment of said mRNA will be oligonucleotide probes, preferably labeled, or oligonucleotide primers.

Said probes or primers may, for example, be designed based on the sequence SEQ ID No.1 as described in U.S. Pat. No. 5,861,274 (cDNA encoding the PPARγ receptor).

A specific hybridization means that the hybridization is carried out under high stringency conditions, in particular under conditions of temperature and of ionic strength such that they allow the hybridization between two DNA fragments which are complementary overall to be maintained.

By way of illustration, high stringency conditions for the hybridization step for the purposes of defining the nucleotide sequences described above are advantageously as follows.

The hybridization is carried out at a preferred temperature of 65° C., in the presence of 6×SSC buffer, 5× Denhardt's solution, 0.5% SDS and 100 μg/ml of salmon sperm DNA.

1×SSC corresponds to 0.15M NaCl and 0.05M of sodium citrate and a 1× Denhardt's solution corresponds to 0.02% Ficoll, 0.02% of polyvinylpyrrolidone and 0.02% of bovine serum albumin.

The washing steps can, for example, be carried out for 5 to 30 min at 65° C., in a 2×SSC or 1×SSC buffer and with 0.1% SDS.

The high stringency conditions described above for a polynucleotide of defined length will be adjusted by those skilled in the art for oligonucleotides which are longer or shorter, according to the teaching of J. Sambrook et al., Molecular cloning, A Laboratory Manual, Cold Spring Harbor Laboratory Press, Cold Spring Harbor, N.Y., 1989.

The oligonucleotide probes or primers, and in general the nucleic acid sequences according to the invention, will be a minimum of 15 bases in length and fragments of at least 20, 25, 30 or 50 bases will be preferred.

The techniques for quantifying and/or localizing a nucleic acid sequence, and which may be used in the methods of the invention, are well known to those skilled in the art and will not be developed in depth here. Mention may be made, without being limited thereto, of the techniques described below.

In general, the specific detection, localization (distribution) and/or assaying of the mRNA of the PPARγ gene, in the biological sample, will comprise the following steps for the in situ hybridization (steps b) and c) of the methods according to the invention):

bringing an oligonucleotide probe, where appropriate labeled, into contact with the biological sample, the mRNA contained in the biological sample having, where appropriate, been made accessible to hybridization beforehand, under conditions which allow the probe to hybridize to the mRNA contained in the biological sample;

detecting, localizing and/or assaying the hybrid formed between the oligonucleotide probe and the mRNA of the biological sample.

When the mRNA of the PPARγ gene is quantified using a total RNA extract of the sample taken, where appropriate enriched in mRNA, the quantification of the mRNA of the PPARγ gene in the extract prepared comprises, in general, the following steps of the “quantitative RT-PCR” method (steps b) and c) of the methods according to the invention):

obtaining a cDNA from the mRNA of the PPARγ gene using specific primers and a reverse transcriptase;

specifically amplifying the PPARγ cDNA obtained by a “PCR” (polymerase chain reaction) or “PCR-like” method using a specific pair of primers;

quantitatively analyzing the amplification products.

Mention may also be made of another method for quantifying the mRNA of the PPARγ gene using a total RNA extract of the sample taken, where appropriate enriched in mRNA, said method comprising the following steps (steps b) and c) of the methods according to the invention):

bringing a first oligonucleotide probe specific for the mRNA of the PPARγ gene or, where appropriate, for its amplification products, immobilized on a support, into contact with a total RNA extract of the sample or, where appropriate, with the PCR products of the mRNA of the PPARγ gene, obtained using specific primers, under conditions which allow the first probe to hybridize to the RNA of said sample or, where appropriate, to said PCR products;

bringing the hybrid formed between said first probe immobilized on a support and said RNAs or, where appropriate, said PCR products, where appropriate after elimination of the nucleic acids which have not hybridized with the first probe, into contact with a second oligonucleotide probe, in particular labeled, capable of hybridizing to the mRNA of the PPARγ gene or, where appropriate, to its PCR products, and then eliminating the second probes which are not hybridized; and

quantitatively analyzing the triplexes thus formed.

The term “PCR-like” will be intended to denote all the methods using direct or indirect reproductions of nucleic acid sequences, or else in which the labeling systems have been amplified. These techniques are of course known. In general, they involve amplification of the DNA by a polymerase; when the sample of origin is an RNA, it is advisable to carry out a reverse transcription beforehand. A large number of methods currently exist which allow this amplification, for example the methods termed NASBA “Nucleic Acid Sequence Based Amplification”, TAS “Transcription based Amplification System”, LCR “Ligase Chain Reaction”, “Endo Run Amplification” (ERA), “Cycling Probe Reaction” (CPR), and SDA “Strand Displacement Amplification” which are well known to those skilled in the art. Also known to those skilled in the art are the techniques for quantifying nucleic acids in which, for example, the nucleic acid to be quantified is amplified by a PCR-type method and in the presence of a standard nucleic acid which is of the same length, which is a known amount and which is capable of hybridizing to the same primers as the target nucleic acid.

The invention also comprises a method according to the invention, characterized in that the quantification or the visualization (the image) of the signal obtained in step b) and, where appropriate, the distribution of the product from expression of said PPARγ gene between said cells of the intestinal epithelium and of the intestinal lamina propria on the patient's sample are compared with those obtained for a control sample (or control).

In a preferred embodiment, said control sample is a sample of bowel tissue taken from a healthy patient or from a patient suffering from a known chronic inflammatory bowel disease, preferably chosen from Crohn's disease (CD) and ulcerative colitis (UC).

The present invention relates quite particularly and very preferably to an in vitro method for diagnosing, by immunohistochemistry, ulcerative colitis (UC) in a patient who may be suffering from or who may progress toward an inflammatory bowel disease, using a histological section of a sample of bowel tissue taken beforehand from this patient, said sample comprising at least cells of the intestinal epithelium, characterized in that it comprises the following step:

a) bringing said histological section into contact with a compound capable of binding specifically to the PPARγ gene translation product, under conditions which allow the formation of a complex between said compound and said translation product, said compound being labeled or being capable of being labeled in order to obtain a labeling or a signal representative of the amount of said translation product present in the sample and, where appropriate, making it possible to localize said PPARγ gene translation product; and

b) quantifying or visualizing the intensity of the labeling or of the signal obtained in step a) in the cells of the intestinal epithelium.

The inventors have in fact demonstrated, most surprisingly, that it is possible to diagnose, with very high sensitivity and a specificity close to 100%, ulcerative colitis (UC) in a patient by immunohistochemistry carried out on a histological section of a sample of bowel tissue taken from this patient, this sample comprising at least cells of the intestinal epithelium, and compared in particular to the sensitivity and/or the specificity obtained with a diagnostic test carried out by quantitative PCR on the PPARγ gene transcription product or with a diagnostic test carried out by Western blotting on the PPARγ gene translation product using a sample in which the cells of the intestinal epithelium have not been correctly isolated. In fact, the inventors were able to demonstrate, for the first time, that only observation of the deficiency in expression of the PPARγ gene translation product in the cells of the intestinal epithelium made it possible to perform so sensitive and specific a diagnosis. Such a diagnostic test carried out by immunohistochemistry may readily be carried out commercially, and relatively inexpensively, compared to a test carried out by quantitative PCR or Western blotting in which it will have to be necessary to isolate the cells of the intestinal epithelium of the sample taken in order to avoid any contamination with neighboring nonepithelial cells in the sample. In fact, any presence of nonepithelial cells during the complete extraction of the mRNA encoding PPARγ (for the PCR test) or during the extraction of total proteins (for the Western blotting test) will be liable to increase the amount of PPARγ gene transcription or translation product. Such a prior isolation of the cells of the intestinal epithelium of the sample taken, in order to attempt to carry out a test by quantitative PCR or by Western blotting for diagnosing ulcerative colitis (UC) in a patient, with the sensitivity and specificity which may be obtained by immunohistochemistry is difficult to perform and can hardly be envisioned from a commercial point of view.

In an embodiment also preferred, the invention relates to an in vitro method for diagnosing, by immunohistochemistry, ulcerative colitis (UC) according to the present invention, characterized in that the quantification or the visualization of the intensity of the signal obtained in step a) is compared to that obtained for a histological section derived from a sample from a healthy patient.

In an embodiment also preferred, the invention relates to an in vitro method for diagnosing, by immunohistochemistry, ulcerative colitis (UC) according to the present invention, characterized in that, in step b), a decrease in the intensity of the labeling or of the signal obtained in step a) in the cells of the intestinal epithelium derived from the sample from the patient who may be suffering from or who may progress toward an inflammatory bowel disease is, where appropriate, observed, compared to those derived from a sample from a healthy patient.

In an embodiment also preferred, the invention relates to an in vitro method for diagnosing, by immunohistochemistry, ulcerative colitis (UC) according to the present invention, characterized in that said sample is a biopsy of bowel tissue taken from the patient's colon or small intestine.

In an embodiment also preferred, the invention relates to an in vitro method for diagnosing, by immunohistochemistry, ulcerative colitis (UC) according to the present invention, characterized in that said sample is a biopsy of bowel tissue taken from the patient's colon.

In an embodiment also preferred, the invention relates to an in vitro method for diagnosing, by immunohistochemistry, ulcerative colitis (UC) according to the present invention, characterized in that said sample from the patient who may be suffering from or who may progress toward an inflammatory bowel disease is a sample of bowel tissue taken from a healthy or damaged mucosa of the patient.

In an embodiment also preferred, the invention relates to an in vitro method for diagnosing, by immunohistochemistry, ulcerative colitis (UC) according to the present invention, characterized in that said compound capable of binding specifically to said PPARγ gene translation product in step a) is chosen from the following compounds:

a monoclonal or polyclonal antibody, where appropriate labeled, directed against the PPARγ receptor; and

a natural or synthetic, where appropriate labeled, ligand which is an agonist or antagonist for the PPARγ receptor.

In another aspect, the invention relates to a diagnostic composition, characterized in that it comprises a monoclonal or polyclonal antibody, where appropriate labeled, directed against the PPARγ receptor.

The invention also relates to a diagnostic composition, characterized in that it comprises a natural or synthetic, where appropriate labeled, ligand which is an agonist or antagonist for the PPARγ receptor, said ligands preferably being chosen from those mentioned above.

The invention also relates to a diagnostic composition, characterized in that it comprises a nucleic acid sequence, where appropriate labeled, capable of hybridizing specifically with a fragment of the mRNA or of the cDNA encoding the PPARγ receptor, preferably comprising at least 15, 20, 25, 30, 50 or 75 bases.

In another aspect, a subject of the present invention is the use of a compound chosen from:

a monoclonal or polyclonal antibody, where appropriate labeled, directed against the PPARγ receptor;

a natural or synthetic, where appropriate labeled, ligand which is an agonist or antagonist for the PPARγ receptor; or

a nucleic acid sequence, where appropriate labeled, capable of hybridizing specifically with a fragment of the mRNA or of the cDNA encoding the PPARγ receptor,

for preparing a composition intended for diagnosing or for giving a prognosis for inflammatory bowel disease in a patient.

Preferably, the use according to the invention is characterized in that said inflammatory bowel disease is a chronic disease, in particular Crohn's disease (CD) or ulcerative colitis (UC).

Also preferably, the use according to the invention is characterized in that said inflammatory bowel disease is an acute bowel disease in a patient.

In another even more preferred aspect, a subject of the present invention is the use of a compound chosen from:

a monoclonal or polyclonal antibody, where appropriate labeled, directed against the PPARγ receptor; or

a natural or synthetic, where appropriate labeled, ligand which is an agonist or antagonist of the PPARγ receptor,

for preparing a composition intended for diagnosing or for giving a prognosis for ulcerative colitis (UC).

The kits or packs for diagnosing inflammatory bowel disease, in particular chronic inflammatory bowel disease such as Crohn's disease (CD) or ulcerative colitis (UC), are advantageously part of the invention.

In a final aspect, the present invention thus relates to a kit or pack for diagnosing or for giving a prognosis for inflammatory bowel disease in a patient, characterized in that it comprises:

a) at least one of the compounds chosen from the following compounds:

a monoclonal or polyclonal antibody, where appropriate labeled, directed against the PPARγ receptor;

a natural or synthetic, where appropriate labeled, ligand which is an agonist or antagonist for the PPARγ receptor; or

a nucleic acid sequence, where appropriate labeled, capable of hybridizing specifically with a fragment of the mRNA encoding the PPARγ receptor;

b) where appropriate, the reagents for making up the medium suitable for the formation of a complex between said compound as defined in a) and a PPARγ gene expression product;

c) where appropriate, the reagents for quantifying or visualizing complexes possibly formed between said compound as defined in a) and a PPARγ gene expression product;

d) where appropriate, a control sample, preferably a total protein extract of a sample of bowel tissue or a section of bowel tissue taken from a healthy patient and/or from a patient suffering from an inflammatory bowel disease of known type.

Finally, the present invention preferably relates to a kit or pack for diagnosing, by immunohistochemistry, ulcerative colitis (UC) in a patient who may be suffering from or who may progress toward an inflammatory bowel disease, characterized in that it comprises:

a) at least one of the compounds chosen from the following compounds:

a monoclonal or polyclonal antibody, where appropriate labeled, directed against the PPARγ receptor; or

a natural or synthetic, where appropriate labeled, ligand which is an agonist or antagonist for the PPARγ receptor; and

b) where appropriate, the reagents for making up the medium suitable for the formation of a complex between said compound as defined in a) and the PPARγ gene translation product;

c) where appropriate, the reagents for quantifying or visualizing complexes possibly formed between said compounds as defined in a) and the PPARγ gene translation product;

d) where appropriate, a control histological section of bowel tissue taken from a healthy patient and/or from a patient suffering from an inflammatory bowel disease of known type, on which control histological section(s), where appropriate, the immunohistochemical labeling of the PPARγ gene translation product has been carried out beforehand;

e) where appropriate, an instruction sheet showing pictures of histological sections obtained after immunohistochemical labeling of the PPARγ gene translation product for control histological sections of bowel tissue derived from samples from a healthy patient, from a patient suffering from ulcerative colitis and/or from a patient suffering from Crohn's disease.

The kits or packs for diagnosing inflammatory bowel disease according to the present invention may also comprise an instruction sheet showing the user the levels and/or distribution of the PPARγ gene expression products expected for samples derived from a healthy patient and from a patient suffering from inflammatory bowel disease, in particular chronic inflammatory bowel disease such as Crohn's disease (CD) or ulcerative colitis (UC) as shown in FIGS. 1A, 1B, 2A and 2B below.

Other characteristics and advantages of the invention emerge in the remainder of the description, with the examples and the figures for which the legends are given below.

### FIGURE LEGENDS

FIGS. 1A and 1B

FIG. 1A represents a Western blot carried out for a protein extract obtained from a biopsy from a healthy patient (control, three representative patients), from a patient suffering from Crohn's disease (marked CD, three representative patients) or from a patient suffering from ulcerative colitis (marked UC, three representative patients). This Western blot demonstrates a decrease in the intensity of the band corresponding to the PPARγ protein (of approximately 53 kDa) for the patients suffering from Crohn's disease compared to the healthy control, which decrease is even more accentuated for the patients suffering from ulcerative colitis.

FIG. 1B represents, for each sample tested (18 for the samples from a healthy patient (control), 18 for the samples from a patient suffering from CD and 15 for the samples from a patient suffering from UC), the amount of PPARγ protein expressed, the amount being expressed as optical density per 50 μg of total protein.

FIGS. 2A, 2B and 2C

FIGS. 2A to 2C represent a histological section derived from a bowel biopsy taken from the colon, in which the PPARγ receptors have been immunolabeled. FIG. 2A represents the histological section from a healthy patient (control), FIG. 2B represents that from a patient suffering from CD and FIG. 2C represents that from a patient suffering from UC. In these figures, the following may in particular be observed:

FIG. 2A: a high density of labeling on the epithelial cells for the control sample with a low density of labeling for the cells of the lamina propria;

FIG. 2B: a high density of labeling on the epithelial cells, with, however, a decrease in the intensity of the labeling, and also a considerable density of labeling on the cells of the lamina propria; and

FIG. 2C: a lack of labeling (or very faint labeling) on the epithelial cells and on the cells of the lamina propria.

### EXAMPLES

Patients

All the patients analyzed gave their consent for this study, for which the conditions were approved by local ethics committees. The diagnosis of Crohn's disease and of ulcerative colitis was established according to the usual criteria (Rutgeerts, P., et al., Gastroenterology 99:956-963, 1990).

### Example 1

### Quantification of PPARγ in the Colon by Western Blotting

Total protein extracts are obtained by homogenizing colon biopsies in a lysis buffer consisting of PBS (phosphate buffered saline) with 1% of NP-40 (™Nonidet P40), 0.5% of sodium deoxycholate, 0.1% of sodium dodecyl sulfate and a conventional cocktail of protease inhibitors (Fajast, L., et al., J. Biol. Chem., 272:18779-18789, 1997).

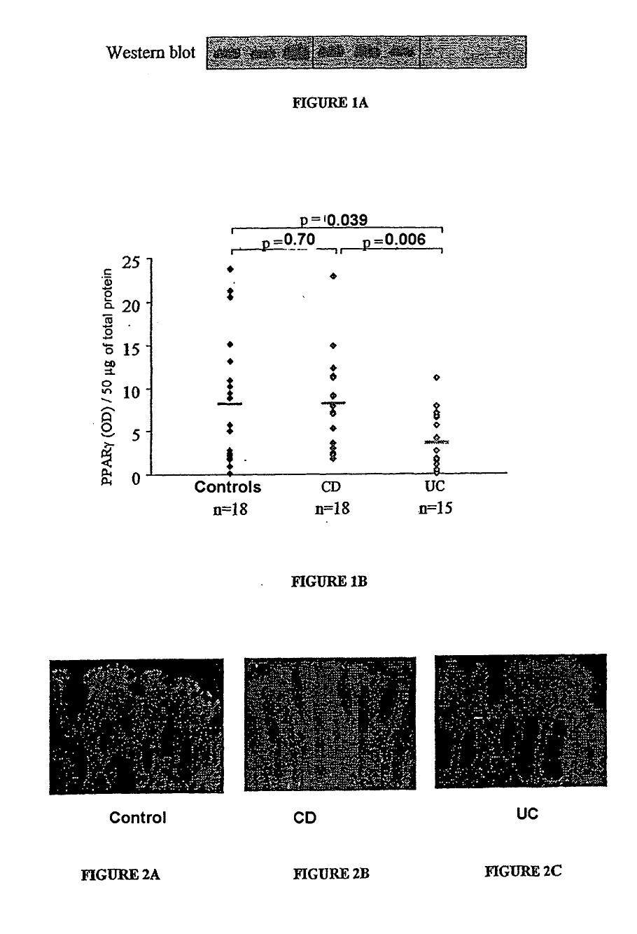

The separation of total proteins (50 μg), the transfer onto PVDF (polyvinylidene difluoride) membrane (cf. FIG. 1A) and the immunodetection of PPARγ by incubation of the membrane with a rabbit polyclonal antiserum for 12 hours (1/500 dilution, TEBU, Le Perray en Yvelines, France) were carried out as previously described (Lefebvre, et al., 1999).

The complex is revealed by chemiluminescence (ECL, Amersham, UK), as indicated in the supplier's protocol. The results are expressed in units of optical density (OD) per 50 μg of total proteins (cf, FIG. 1B).

### Example 2

### Immunolabeling of PPARγ in the colon

The colon biopsies are fixed in 4% paraformaldehyde, embedded in paraffin and sectioned at 4 micrometers for the immunohistochemistry and the immunofluorescence.

The sections are preincubated for 30 minutes at ambient temperature in a blocking solution containing avidin D and biotin (™Blocking Kit, SP2001, Vector Laboratories, Burlingame, Calif., USA). They are then exposed to a rabbit polyclonal antiserum directed against PPARγ (1/50 dilution, WAK-CHEMIE, Bad Soden, Germany) for two hours at ambient temperature. The sections are washed in PBS containing 0.05% of ™Triton X-100 and incubated with a biotinylated goat anti-rabbit secondary antibody (1/500 dilution for 30 minutes, Dako, Trappes, France).

The immunocomplex is detected by virtue of avidinbiotin coupled to peroxidase (™ABCOMPLEX/HRP, Dako, Trappes, France) and revealed using 3,3′-diaminobenzidine (DAB, Dako, Trappes, France). As negative control, the primary antibody is replaced with an irrelevant rabbit serum.

Statistical Methods

The comparisons of the means±SEM of the amounts of PPARγ receptor protein and mRNA between the patients suffering from UC and from CD and the control patients were analyzed by the Kruskal-Wallis nonparametric ANOVA test. The differences are judged to be statistically different for a p<0.05.

### Example 3

### Results

Using Western blotting (cf. FIG. 1A) and immunohistochemistry (cf. FIGS. 2A to 2C) techniques, a deficiency in expression of the peroxysome proliferator-activated receptor PPARγ was demonstrated in the intestinal epithelial cells, for the first time, in the healthy or damaged colonic mucosa especially in patients suffering from UC and, to a lesser degree, in the patients suffering from CD (visible in the intensity of the labeling by immunohistochemistry). This deficiency in expression is thus readily demonstrated in patients suffering from UC (cf. FIGS. 1B and 2C). This deficiency also appears to be significant for CD and can also be demonstrated by observing the intensity of the immunolabeling by immunohistochemistry (cf. FIG. 2B).

As regards CD, the immunohistochemistry technique demonstrates a gain in expression of the PPARγ receptor in the cells of the lamina propria, whereas the expression in these cells is not apparent, or is only very weakly apparent, for UC or for healthy patients.

As regards the cells of the lamina propria, a deficiency in expression may also be noted for the patients suffering from UC compared to the healthy controls.

The detection of PPARγ in the colonic mucosa is a new marker for making a rapid diagnosis of UC and/or of CD. This detection is possible by performing biopsies carried out on healthy or damaged mucosa during an endoscopic examination which can be limited to exploration of the first 20 cm of the colon in the region of the rectum and the sigmoid.

The tests carried out on all the samples analyzed show that the sensitivity and the specificity are 100% in CD and UC.

The techniques used are simple and can be carried out routinely by all anatomy/pathology laboratories.

Among the other advantages of the method according to the invention, mention may in particular be made of the fact that:

the biopsies can be performed both on healthy mucosa and on damaged mucosa, during periods of quiescence of the disease or during attacks; and that

the rectal biopsies require only a short endoscopy, without general anesthetic and without colonic preparation.

The invention also relates to a diagnostic device comprising means adapted for carrying out the various steps of the method described above.

1. An in vitro method for determining the amount of product from expression of the gene encoding PPARγ (PPARγ gene) on a sample of bowel tissue, said sample comprising at least cells of the intestinal epithelium and/or of the intestinal lamina propria, characterized in that it comprises the following steps:
a) taking a sample of bowel tissue from a patient who may be suffering from or who may progress toward an inflammatory bowel disease, said sample comprising at least cells of the intestinal epithelium and/or of the intestinal lamina propria;
b) bringing the PPARγ gene expression product into contact with a compound capable of binding specifically to said PPARγ gene expression product, under conditions which allow the formation of a complex between said compound and said expression product, said compound being labeled or being capable of being labeled in order to obtain a signal representative of the amount of said expression product present in the sample and, where appropriate, making it possible to localize said expression product; and
c) quantifying or visualizing the signal obtained in step b).

2. The method as claimed in claim 1, characterized in that said sample of bowel tissue comprises at least cells of the intestinal epithelium and of the intestinal lamina propria, and in that it also uses a step for comparing the distribution of the product from expression of said PPARγ gene between said cells of the intestinal epithelium and of the intestinal lamina propria on the patient's sample.

3. The method as claimed in claim 1, characterized in that said sample is a biopsy of bowel tissue taken from the patient's colon or small intestine.

4. The method as claimed in claim 1, characterized in that said sample is a biopsy of bowel tissue taken from the patient's colon.

5. The method as claimed in claim 1, characterized in that said product from expression of said PPARγ gene is the product of translation of said PPARγ gene, and in that the compound capable of binding specifically to said PPARγ gene expression product in step b) is chosen from the following compounds:
a monoclonal or polyclonal antibody, where appropriate labeled, directed against the PPARγ receptor; and
a natural or synthetic, where appropriate labeled, ligand which is an agonist or antagonist for the PPARγ receptor.

6. The method as claimed in claim 5, characterized in that, prior to step b):
the total proteins are extracted from the sample taken; and then
separated and transferred onto membrane (Western blotting); and
in that, in step c), the signal obtained in step b) is quantified.

7. The method as claimed in claim 5, characterized in that, prior to step b):
a histological section of the sample taken is prepared, said sample taken having been, where appropriate, fixed in a solution of paraformaldehyde or formaldehyde and embedded in paraffin; and
in that, in step c), the signal obtained in step b) is visualized.

8. The method as claimed in claim 1, characterized in that said product from expression of said PPARγ gene is the mRNA product of transcription of said PPARγ gene, and in that the compound capable of binding specifically to said PPARγ gene expression product in step b) is chosen from the nucleic acid sequences, where appropriate labeled, capable of hybridizing specifically with a fragment of said mRNA.

9. The method as claimed in one of claims 1 to 8, characterized in that the quantification or the visualization of the signal obtained in step b) and, where appropriate, the distribution of the product from expression of said PPARγ gene between said cells of the intestinal epithelium and of the intestinal lamina propria on the patient's sample are compared with those obtained for a control sample.

10. The method as claimed in claim 9, characterized in that said control sample is a sample of bowel tissue taken from a healthy patient or a patient suffering from a chronic inflammatory bowel disease, preferably chosen from Crohn's diease (CD) and ulcerative colitis (UC).

11. An in vitro method for diagnosing, by immunohistochemistry, ulcerative colitis (UC) in a patient who may be suffering from or who may progress toward an inflammatory bowel disease, using a histological section of a sample of bowel tissue taken beforehand from this patient, said sample comprising at least cells of the intestinal epithelium, characterized in that it comprises the following step:
a) bringing said histological section into contact with a compound capable of binding specifically to the PPARγ gene translation product, under conditions which allow the formation of a complex between said compound and said translation product, said product being labeled or being capable of being labeled in order to obtain a labeling or a signal representative of the amount of said translation product present in the sample and, where appropriate, making it possible to localize said PPARγ gene translation product; and
b) quantifying or visualizing the intensity of the labeling or of the signal obtained in step a) in the cells of the intestinal epithelium.

12. The in vitro method for diagnosing, by immunohistochemistry, ulcerative colitis (UC) as claimed in claim 11, characterized in that the quantification or the visualization of the intensity of the signal obtained in step a) is compared to that obtained for a histological section derived from a sample from a healthy patient.

13. The in vitro method for diagnosing, by immunohistochemistry, ulcerative colitis (UC) as claimed in claim 11 or 12, characterized in that, in step b), a decrease in the intensity of the labeling or of the signal obtained in step a) in the cells of the intestinal epithelium derived from the sample from the patient who may be suffering from or who may progress toward an inflammatory bowel disease is, where appropriate, observed, compared to those derived from a sample from a healthy patient.

14. The method as claimed in one of claims 11 to 13, characterized in that said sample is a biopsy of bowel tissue taken from the patient's colon or small intestine.

15. The method as claimed in claim 14, characterized in that said sample is a biopsy of bowel tissue taken from the patient's colon.

16. The method as claimed in one of claims 11 to 15, characterized in that said sample from the patient who may be suffering from or who may progress toward an inflammatory bowel disease is a sample of bowel tissue taken from a healthy or damaged mucosa of the patient.

17. The method as claimed in one of claims 11 to 16, characterized in that said compound capable of binding specifically to said PPARγ gene translation product in step a) is chosen from the following compounds:
a monoclonal or polyclonal antibody, where appropriate labeled, directed against the PPARγ receptor; and
a natural or synthetic, where appropriate labeled, ligand which is an agonist or antagonist for the PPARγ receptor.

18. A diagnostic composition, characterized in that it comprises a monoclonal or polyclonal antibody, where appropriate labeled, directed against the PPARγ receptor.

19. A diagnostic composition, characterized in that it comprises a natural or synthetic, where appropriate labeled, ligand which is an agonist or antagonist for the PPARγ receptor.

20. A diagnostic composition, characterized in that it comprises a nucleic acid sequence, where appropriate labeled, capable of hybridizing specifically with a fragment of the mRNA encoding the PPARγ receptor.

21. The use of a compound chosen from:
a monoclonal or polyclonal antibody, where appropriate labeled, directed against the PPARγ receptor;
a natural or synthetic, where appropriate labeled, ligand which is an agonist or antagonist for the PPARγ receptor; or
a nucleic acid sequence, where appropriate labeled, capable of hybridizing specifically with a fragment of the mRNA encoding the PPARγ receptor,
for preparing a composition intended for diagnosing or for giving a prognosis for inflammatory bowel disease in a patient.

22. The use of a compound as claimed in claim 21, characterized in that said inflammatory bowel disease is a chronic disease.

23. The use of a compound as claimed in claim 22, characterized in that said chronic inflammatory bowel disease is Crohn's disease (CD) or ulcerative colitis (UC).

24. The use of a compound as claimed in claim 21, characterized in that said inflammatory bowel disease is an acute bowel disease in a patient.

25. The use of a compound chosen from:
a monoclonal or polyclonal antibody, where appropriate labeled, directed against the PPARγ receptor; or
a natural or synthetic, where appropriate labeled, ligand which is an agonist or antagonist of the PPARγ receptor,
for preparing a composition intended for diagnosing or for giving a prognosis for ulcerative colitis (UC) in a patient.

26. A kit or pack for diagnosing or for giving a prognosis for inflammatory bowel disease in a patient, characterized in that it comprises:
a) at least one of the compounds chosen from the following compounds:
a monoclonal or polyclonal antibody, where appropriate labeled, directed against the PPARγ receptor;
a natural or synthetic, where appropriate labeled, ligand which is an agonist or antagonist for the PPARγ receptor; or
a nucleic acid sequence, where appropriate labeled, capable of hybridizing specifically with a fragment of the mRNA encoding the PPARγ receptor;
b) where appropriate, the reagents for making up the medium suitable for the formation of a complex between said compound as defined in a) and a PPARγ gene expression product;
c) where appropriate, the reagents for quantifying or visualizing complexes possibly formed between said compound as defined in a) and a PPARγ gene expression product;
d) where appropriate, a control sample, preferably a total protein extract of a sample of bowel tissue or a section of bowel tissue taken from a healthy patient and/or from a patient suffering from an inflammatory bowel disease of known type.

27. A kit or pack for diagnosing, by immunohistochemistry, ulcerative colitis (UC) in a patient who may be suffering from or who may progress toward an inflammatory bowel disease, characterized in that it comprises:
a) at least one of the compounds chosen from the following compounds:
a monoclonal or polyclonal antibody, where appropriate labeled, directed against the PPARγ receptor; or
a natural or synthetic, where appropriate, ligand which is an agonist or antagonist for the PPARγ receptor; and
b) where appropriate, the reagents for making up the medium suitable for the formation of a complex between said compound as defined in a) and the PPARγ gene translation product;
c) where appropriate, the reagents for quantifying or visualizing complexes possibly formed between said compound as defined in a) and the PPARγ gene translation product;
d) where appropriate, a control histological section of bowel tissue taken from a healthy patient and/or from a patient suffering from an inflammatory bowel disease of known type, on which control histological section(s) the immunohistochemical labeling of the PPARγ gene translation product has been carried out beforehand;
e) where appropriate, an instruction sheet showing pictures of histological sections obtained after immunohistochemical labeling of the PPARγ gene translation product for control histological sections of bowel tissue derived from samples from a healthy patient, from a patient suffering from ulcerative colitis and/or from a patient suffering from Crohn's disease.
